(19) United States
(12) Patent Application Publication
(10) Pub. No.: US 2020/0397369 A1
(43) Pub. Date: Dec. 24, 2020

(54) Device and method for treating glioblastoma and other malignant tumors of the brain and central nervous system with ultraviolet radiation

(71) Applicant: Keith Reed Greenbaum, Providence, RI (US)

(72) Inventor: Keith Reed Greenbaum, Providence, RI (US)

(21) Appl. No.: 16/501,871

(22) Filed: Jun. 22, 2019

(51) Int. Cl. A61B 5/00; A61N 5/06; G16H 50/50

(52) U.S. Cl. CPC A61B 5/4848; A61N 2005/0661; G16H 50/50; A61N 5/0613

(57) ABSTRACT

This patent application presents a device and method for treating glioblastoma and other malignant tumors of the brain and central nervous system with ultraviolet radiation. The evidence for this possible treatment comes from a large statistical correlation between the incidence of these cancers and the strength of ultraviolet reaching the ground in that location. The weaker the ultraviolet, the more such tumors. The device uses the yearly intensity of ultraviolet from Hawaii, the place with the strongest ultraviolet and the lowest incidence rate for such malignant tumors. The method entails exposure to the ultraviolet in the device for twenty minutes a day, three times a week. The hope is that people with these malignant tumors will live longer than those not undergoing this treatment.

This patent application presents a device and method for treating glioblastoma and other malignant tumors of the brain and central nervous system with ultraviolet radiation. The need is great for better treatments for these deadly tumors in light of the short amount of time until death occurs. For glioblastoma, the main malignant tumor in this category (with the highest number of cases), the median survival time is about a year.

While this proposed device and method has not yet been tried, it is based on a statistical result, a correlation, between two sets of numbers which is surprisingly high. The incidence rate by state for these brain and central nervous system cancers can be linked to the intensity of ultraviolet reaching the ground in that state over the course of the year. The stronger the ultraviolet in a state, the fewer the people in that state (per population size) who have these cancerous tumors. This connection is a correlation of over 0.7 and is negative in direction. In light of this connection, I posit ultraviolet as the basis of a device and method for treating these cancers.

Now, we need some more exact detail about each set of numbers. The incidence (the number of people having the disease) is expressed as a yearly rate (for the size of the population) by state including people of all ages. These numbers are age adjusted. This rate of malignant tumors of the brain and central nervous system is from the CBTRUS Statistical Report, from 2016, Table 5 and includes a yearly incidence rate for the years 2009-2013. (doi:10.1093/neuonc/now207) from an article by Quinn T. Ostrom et al. 2016. CBTRUS stands for Central Brain Tumor Registry of the United States with both public and private funding. These numbers are included here further along in Table 1.

The other set of numbers is the yearly UV Index for the largest city in each US state over the course of the year 1996, a version of the UV index created especially for the research in my unpublished book called “Cancer in America” copyright 2017 by Keith Reed Greenbaum. These numbers were created by an expert in the UV Index at the National Weather Service of NOAA years ago by one of their scientists Craig Long. The number for each state is an expression of the intensity of ultraviolet reaching the ground in sunlight at “solar noon” at that location over the course of the entire year.

The daily estimate uses weather information from the satellite to make a prediction of the intensity of ultraviolet reaching the ground the following afternoon. Mr. Long converted this daily estimate to one that summarizes the entire year in one number for the largest city of the state, used here to represent the entire state.

In trying to build a model to explain the pattern of variation from state to state in the incidence of malignant tumors of the brain and the central nervous system, this one item explained more than half of the pattern of variation between states (adjusted r square=0.504). For one item to explain half the pattern is unusual particularly given the fact that these numbers are already adjusted for age.

Furthermore, the “zero order” correlation between this yearly UV Index score for the state and the incidence of these malignant tumors in that state was negative −0.717. The stronger the intensity of ultraviolet reaching the ground as measured in this yearly version of the UV Index, the lower the incidence rate of these cancers.

The UV Index is an unusually good measure of the intensity of ultraviolet reaching the ground in sunlight because it is a weighted measure which weights the stronger B portion in order to better capture the effect of this part of sunlight on human skin.

On the other hand, this general macro measure of ultraviolet intensity is not able to measure a particular individual's exposure to ultraviolet. Nevertheless, while I posit that exposure is involved as an explanation for the result, the strong correlation must be primarily due to variations in the intensity of ultraviolet itself.

The result can also be stated the other way around. If more people in a state have malignant brain and central nervous system tumors the weaker the ultraviolet in sunlight, then perhaps this portion of sunlight might serve as a treatment for this disease.

This is the evidence foundation behind the device and method for treating such cancers. A device that “artificially” emits ultraviolet radiation might be effective for extending the lifespan of people who have such malignant tumors.

Here, I can link this patent application to another slightly earlier one where I propose the same device and method for treating cancers in eight death categories including lung, melanoma, ovarian, uterine, urinary (including bladder) lymphoma (adult) and prostate. That is called “A Device and Method for Treating Eight Cancers with Ultraviolet Radiation”. Filing date Apr. 15, 2019. Keith Reed Greenbaum. U.S. application Ser. No. 16/501,431.

I should point out that the statistical models for those eight cancers used state death rates from the Center for Disease Control, mostly from the Health Data Interactive table. In contrast, this data on malignant brain and central nervous system tumors are based on incidence rates (rather than death rates) and are from a separate source, the CBTRUS, Central Brain Tumor Registry of the United States.

With a few comments, it is important to briefly integrate this new finding about malignant brain and central nervous system tumors into the broader picture including the other eight cancers (based on death categories).

First, the incidence pattern between states here shares in common with the death rates for the other cancers a geographic pattern of north. True across the United States, located, of course, in the Northern Hemisphere, the more north the state happens to be, the higher incidence of brain and central nervous system tumors and the higher death rate from the eight cancers.

Second, for all of the cancers, it is possible to build a statistical model explaining the difference between states by showing a connection with the intensity of ultraviolet reaching the ground in sunlight in that place. For all of the cancers, the direction of the connection is the same. The more intense the ultraviolet, the fewer the deaths from the eight cancers and the fewer people (per population size) who have these malignant brain and central nervous system tumors.

Third, for both the incidence of these malignant brain and central nervous system tumors and a few of the other cancers, it is also possible to show a connection with another environmental factor for explaining the pattern between states other than ultraviolet—one that is linked with ultraviolet in a high correlation (negative in direction)—geomagnetism. Generally speaking, the magnetic field strength on the ground in a location increases as we move north in the United States and it also works well for many of the cancers for explaining this northern pattern. The higher the geomagnetism in the largest city of the state, the more cancer.

In a judgement call by myself, particularly given the improbability of a biological effect for such small changes in the size of the magnetic field, I have chosen to highlight the connection with ultraviolet after being able to build a model showing that link. Today, an experimental physicist who has done work with magnetic fields shared with me his view that it is highly unlikely that differences in geomagnetism could be responsible for a geographic pattern of cancer deaths given the small absolute size of Earth magnetic fields. He pointed out to me the good safety profile of MRI scans with magnetic fields that are many times the size of those occurring naturally from inside the Earth.

Finally, in a last minute development, I heard (via email) from the brain cancer registry CBTRUS this morning and received a reference for state death rates from this kind of cancer. These death rate numbers produced an explanatory model that was less definitive than the incidence numbers I present here. The reasons for this different result are unclear. In another judgement call, I, nevertheless, decided to proceed in the belief that the strikingly high correlation between the numbers I present is evidence for the high quality of the incidence numbers I am using here.

Before describing the device and method, I will finish off this foundational section by presenting the actual two sets of numbers that correlated with each other (Table 1 and Table 2). Then, I will follow that with the model showing both the correlation and, above that, a one item model explaining the pattern of differences in the incidence rate between states (Table 3). Lastly, I will use that model to create a “predicted” rate of cancer incidence based on the effect of ultraviolet in order to better understand the potential power of the finding in terms of incidence rates (Table 4) and finally, the number of human lives affected.

Table 1 is the actual brain and central nervous system tumor incidence rate by state the details of which are in the heading of the chart. It goes from a low in Hawaii of 4.86 cases (probably per hundred thousand people) to a high in New Hampshire of 8.55 people (with a malignant tumor of the brain or central nervous system). This is one of the two sets of numbers in the correlation. (District stands for Washington D.C.).

TABLE 1
[birma913] (brain incidence rates malignant 09-13)
Annual incidence rates by state for malignant brain and
central nervous system tumors 2009-2013, all ages,
age adjusted. From lowest to highest rate.

| | |
|---|---|
| Hawaii | 4.86 |
| Nevada | 6.03 |
| New Mexi | 6.05 |
| North Da | 6.56 |
| Louisian | 6.58 |
| Mississi | 6.69 |
| Californ | 6.73 |
| Rhode Is | 6.77 |
| Georgia | 6.84 |
| District | 6.90 |
| Virginia | 6.92 |
| South Ca | 6.94 |
| Arkansas | 7.04 |
| North Ca | 7.06 |
| Maryland | 7.09 |
| Oklahoma | 7.09 |
| Texas | 7.10 |
| West Vir | 7.18 |
| Florida | 7.19 |
| Idaho | 7.19 |
| Illinois | 7.23 |
| Arizona | 7.27 |
| Missouri | 7.28 |
| New York | 7.30 |
| Colorado | 7.32 |
| Alabama | 7.37 |
| Delaware | 7.40 |
| Minnesot | 7.41 |
| South Da | 7.42 |
| Michigan | 7.44 |
| Tennesse | 7.46 |
| Ohio | 7.53 |
| Massachu | 7.54 |
| Indiana | 7.55 |
| Kansas | 7.60 |
| Utah | 7.61 |
| Connecti | 7.63 |
| Montana | 7.64 |
| New Jers | 7.72 |
| Pennsylv | 7.83 |
| Nebraska | 7.98 |
| Vermont | 7.98 |
| Oregon | 8.02 |
| Washing | 8.05 |
| Iowa | 8.14 |
| Kentucky | 8.16 |
| Wyoming | 8.17 |
| Wisconsi | 8.22 |
| Alaska | 8.24 |
| Maine | 8.38 |
| New Hamp | 8.55 |

Source: Table 5, CBTRUS Statistical Report (Central Brain Tumor Registry of the United States), NPCR (National Program of Cancer Registries) and SEER (Surveillance, Epidemiology and End Results program).
From Quinn T. Ostrom et al. 2016 doi:10.1093/neuonc/now207. “Primary Brain and Other Central Nervous System Tumors Diagnosed in the United States in 2009-2013”

Table 2 is the yearly UV Index score for each state during the year 1996, an indicator of the intensity of ultraviolet reaching the ground in the largest city of the state over the course of that year. This set of numbers is from my unpublished book “Cancer in America” (copyright 2017 by Keith Reed Greenbaum). These yearly numbers were created at my request by National Weather Service—NOAA scientist Craig Long whose help I gratefully acknowledge even though he bears no responsibility for the contrarian nature of the result. Alaska has the lowest score of all the states at 1.4, an indicator of the strength of ultraviolet reaching the ground in Anchorage over the year. Hawaii is the state with the highest yearly UV Index score of 9.6 in Honolulu. This is the second set of numbers in the correlation.

TABLE 2
[ultra96] ultraviolet. Average annual UVI
(UV Index) score for the largest city in the state
over the course of the year 1996.

| | |
|---|---|
| Alaska | 1.4 |
| Washing | 2.7 |
| Vermont | 2.9 |
| Oregon | 3.0 |
| Maine | 3.1 |
| North Da | 3.1 |
| New Hamp | 3.2 |
| Minnesot | 3.2 |
| Michigan | 3.3 |
| Wisconsi | 3.3 |
| Massachu | 3.3 |
| Ohio | 3.4 |
| Connecti | 3.4 |
| Rhode Is | 3.4 |
| Illinois | 3.5 |
| South Da | 3.5 |
| Montana | 3.5 |
| New York | 3.6 |
| Indiana | 3.7 |
| Iowa | 3.7 |
| Nebraska | 3.8 |
| Pennsylv | 3.8 |
| Maryland | 3.9 |
| Delaware | 3.9 |
| New Jers | 3.9 |
| Idaho | 4.0 |
| West Vir | 4.0 |
| District | 4.0 |
| Kentucky | 4.1 |
| Missouri | 4.1 |
| Virginia | 4.4 |
| North Ca | 4.6 |
| Utah | 4.7 |
| Wyoming | 4.7 |
| Kansas | 4.7 |
| Arkansas | 4.8 |
| Tennesse | 4.8 |
| Colorado | 4.9 |
| Georgia | 5.1 |
| Oklahoma | 5.2 |
| South Ca | 5.3 |
| Mississi | 5.5 |
| Nevada | 5.7 |
| Alabama | 5.8 |
| Texas | 5.9 |
| Californ | 5.9 |
| Louisian | 5.9 |
| New Mexi | 6.1 |
| Arizona | 6.2 |
| Florida | 6.9 |
| Hawaii | 9.6 |

Table 3 includes below the actual correlation between the above two sets of numbers, the incidence rate for brain and central nervous system tumors and the yearly UV Index score for each state. It shows a correlation of (negative) −0.717″. The stronger the ultraviolet reaching the ground over the year, the fewer people in that state (per population size) who have these malignant tumors.

TABLE 3
One Item Model Explaining the Pattern of Incidence between States
of Malignant Tumors of Brain and Central Nervous System with
the Yearly UV Index for that State. The More Intense the Ultraviolet,
the fewer the People with these Malignant Tumors
Model Summary

| | | | Adjusted | Std. Error of |
|---|---|---|---|---|
| Model | R | R Square | R Square | the Estimate |
| 1 | .717(a) | .514 | .504 | .46548 |

(a)Predictors: (Constant), ULTRA96

Coefficients(a)

| | | Unstandardized | | Standardized | | |
|---|---|---|---|---|---|---|
| | | Coefficients | | Coefficients | | |
| Model | | B | Std. Error | Beta | t | Sig. |
| 1 | (Constant) | 8.875 | .223 | | 39.745 | .000 |
| | ULTRA96 | −.356 | .049 | −.717 | −7.196 | .000 |

(a)Dependent Variable: BIRMA913

Correlations

| | | ULTRA96 | BIRMA913 |
|---|---|---|---|
| ULTRA96 | Pearson Correlation | 1 | −.717(**) |
| | Sig. (2-tailed) | . | .000 |
| | N | 51 | 51 |
| BIRMA913 | Pearson Correlation | −.717(**) | 1 |
| | Sig. (2-tailed) | .000 | . |
| | N | 51 | 51 |

(**)Correlation is significant at the 0.01 level (2-tailed).

The top part of Table 3 is the statistical model, however unusual it is to have a one item model. This is an attempt to explain the pattern between states—why more people have these tumors in some states compared to others (for the same size of the population). The one item is, of course, ultraviolet. The stronger the ultraviolet, the fewer people there are who have these tumors.

As I noted earlier, this one item explains over have of the total pattern (in the box Adjusted R Square=0.504). This result is unusually strong given that the incidence numbers are already adjusted for “age”, older age typically being a large portion of the explanation for cancer because older people are more likely to get cancer, true for this kind as well. The two parts of this Table 3 constitute the main foundation of the statistical evidence linking the incidence of brain and central nervous system tumors with the diminished intensity of ultraviolet in certain parts of the country.

Lastly now is an attempt to quantify the meaning of this relationship more clearly between ultraviolet and these malignant tumors of the brain and central nervous system. We attempt to do this by using the equation from the statistical model to create new incidence numbers that capture the exclusive effect of ultraviolet on incidence rates. The purpose here is to compare this “predicted” incidence rate in the state with the highest ultraviolet, Hawaii, with the state with the lowest ultraviolet intensity, Alaska, in order to better see the size difference between them (which is due exclusively to ultraviolet).

The equation we use is from the statistical model in Table 3, the B column. Brainuv=8.875−(0.356*ultra96). The statistical software program adds the UV Index value for each state from Table 2 when solving the equation.

The result is in Table 4 [brainuv] Brain Ultraviolet. Predicted incidence rate of malignant brain (and central nervous system) tumors based on the actual rate by state, all ages, age adjusted) but using the ultraviolet model to “predict” the rate due exclusively to the effect of ultraviolet. (District stands for Washington D.C.).

TABLE 4
[brainuv]. Brain ultraviolet: Predicted incidence rate
of malignant brain (and central nervous system) tumors
based on the actual rate by state, (all ages, age adjusted)
but using the ultraviolet model to “predict” the rate due
exclusively to the effect of ultraviolet

| | |
|---|---|
| Hawaii | 5.46 |
| Florida | 6.42 |
| Arizona | 6.67 |
| New Mexi | 6.70 |
| Louisian | 6.77 |
| Californ | 6.77 |
| Texas | 6.77 |
| Alabama | 6.81 |
| Nevada | 6.85 |
| Mississi | 6.92 |
| South Ca | 6.99 |
| Oklahoma | 7.02 |
| Georgia | 7.06 |
| Colorado | 7.13 |
| Arkansas | 7.17 |
| Tennesse | 7.17 |
| Kansas | 7.20 |
| Utah | 7.20 |
| Wyoming | 7.20 |
| North Ca | 7.24 |
| Virginia | 7.31 |
| Missouri | 7.42 |
| Kentucky | 7.42 |
| District | 7.45 |
| West Vir | 7.45 |
| Idaho | 7.45 |
| Maryland | 7.49 |
| Delaware | 7.49 |
| New Jers | 7.49 |
| Pennsylv | 7.52 |
| Nebraska | 7.52 |
| Indiana | 7.56 |
| Iowa | 7.56 |
| New York | 7.59 |
| Illinois | 7.63 |
| South Da | 7.63 |
| Montana | 7.63 |
| Rhode Is | 7.66 |
| Ohio | 7.66 |
| Connecti | 7.66 |
| Michigan | 7.70 |
| Massachu | 7.70 |
| Wisconsi | 7.70 |
| Minnesot | 7.74 |
| New Hamp | 7.74 |
| North Da | 7.77 |
| Maine | 7.77 |
| Oregon | 7.81 |
| Vermont | 7.84 |
| Washing | 7.91 |
| Alaska | 8.38 |

In Table 4, the lowest “predicted” incidence rate is in Hawaii at 5.46 cases and the highest “predicted” incidence rate is in Alaska at 8.38 cases. Now we have the information for the comparison we want. Compared with the place with the highest “predicted” number of cases (Alaska), how much lower is the incidence rate in Hawaii (with the fewest number of cases). 8.38/1=5.46/x. 8.38x=5.46. x=5.46/8.38. X=0.6515513. In Hawaii, the place with the most intense ultraviolet, the incidence rate is only some 65 percent as high. 1−0.6515513=0.3484487.

This means there is a reduction in the predicted incidence rate of these malignant tumors of the brain and central nervous system of more than 34.8 percent, due exclusively to the difference in the strength of ultraviolet reaching the ground in the two places.

So this is another way of picturing the size of the ultraviolet effect on the incidence of these tumors. Due to the difference in the strength of ultraviolet, the incidence rate from these tumors (from state to state) goes down by more than a third as ultraviolet becomes more intense.

Almost at the end, there is one more way of showing the size of the ultraviolet effect, however imperfect. We can attempt to convert this reduction in the incidence rate into actual numbers of people who had these deadly tumors in a typical year between 2009-2013, the years included to create the incidence rates. We can obliquely suggest that if the incidence rate could have been reduced by about a third with stronger ultraviolet, that perhaps this is the percent of individuals who might have been saved from this terrible fate.

While I apologize for the imperfections of this comparison, I consider the attempt worthwhile. Using numbers from a more recent year (and these are projected number of deaths, not incidence), 16,640 deaths are expected to occur in 2018 from “primary brain and central nervous system tumors” in the United States. Roughly, we can now use that above number of a one third reduction in the incidence rate (from the earlier years), to do the following. We multiply both together or 16,640*0.3484487=5798.2. Perhaps close to 5800 people might have been saves in the earlier years had the ultraviolet been stronger.

Of course, the evidence presented here attempts to show a connection between the incidence rate of these malignant brain and central nervous system tumors and the intensity of ultraviolet reaching the ground over the course of the year in a location. The stronger the ultraviolet, the lower the incidence rate of these tumors.

While this statistical association, however large in size, does not constitute proof, it suggests that the intensity of ultraviolet might play a causal role in the development of these malignant tumors, a lack of exposure to ultraviolet that is strong enough. This is different than suggesting that ultraviolet has been tried as an artificial version of this connection in a treatment for people who already have these malignant tumors. However different these two things, it does suggest the possibility that an ultraviolet based device and method for treating these malignant brain tumors might have promise for helping these unfortunate people to live longer.

### A DESCRIPTION OF THE DEVICE AND METHOD FOR TREATING GLIOBLASTOMA AND OTHER MALIGNANT BRAIN AND CENTRAL NERVOUS SYSTEM WITH ULTRAVIOLET RADIATION

In this section, I describe the proposed device and method for treating this kind of cancer. It is actually identical to what I describe for the other eight cancers in a different, recent patent application, the details of which I will repeat now. That patent application is called “A Device and Method for Treating Eight Cancers with Ultraviolet Radiation”. Filing date Apr. 15, 2019. Keith Reed Greenbaum. U.S. application Ser. No. 16/501,431. I will make use of cut and paste making changes when necessary.

The key feature of the device is the intensity of ultraviolet which comes from the statistical result showing a link between stronger ultraviolet and a lower incidence of malignant brain and central nervous system tumors in the United States. This would be set to mimic the yearly average for ultraviolet intensity in the place with the strongest sun—Honolulu Hi. Based on a measure of the yearly UVI (UV Index) for American cities in the year 1996 and modified by actual sun measurements on location (in 2018), this proposed setting would maximize the chances for creating a cancer treatment that is both safe and effective.

The radiation would be almost entirely in the ultraviolet range. It would begin at the beginning of the higher energy UVB portion at a value of 280 nanometers. It would continue up through the end of the UV range ending with the weakest wavelength of the UVA range at 400 nanometers.

The key attribute of ultraviolet intensity is the B portion as measured by a Solarmeter Model 6.0 Standard UVB Meter (after milliwatts per square centimeter is converted to watts per square meter) which would be set at 3 watts per square meter. The B portion is foremost because the UV Index itself is a weighted measure which augments the role of the stronger B portion to focus on the effect of sunlight on human skin. This setting is based on an average of actual summer and winter measurements in Hawaii (near Waikiki Beach in Honolulu), this average itself being an attempt to mimic the year-round strength of ultraviolet in this version of the UV Index used in the research.

This setting would maximize the chances of creating a cancer treatment that, while effective, might also be safer because the strength comes from a range that is an average of naturally occurring intensity values. The UV Index value would be between the lower actual measured average value of 6.6 and the UV Index value of about 9.6 from the statistical research, measured with a Solarmeter Model 6.5 UV index Meter.

As the goal would be the minimum exposure necessary to achieve a therapeutic result, the device would include three sizes with the smallest effective version to be preferred. The two smaller versions would make shipping easier. The first would be a facial-style device with dimensions 22 inches wide, 9 inches deep and 14 inches tall. The Medium machine would have dimensions of about 2 feet tall and 1 foot wide. Both smaller machines would have four florescent-style tubes created with the ultraviolet specifications mentioned above. The large machine would be one row of five or six foot long florescent-style tubes preferably in a frame that can be tilted for use standing or lying. There would be a minimum of six such tubes in the row.

For all three size machines, the distance from the patient would be determined by the value of UVB to be set at 3 watts per square meter. The Small unit would be employed on the face but might also be employed in other areas related to the location of the cancer. The same is true for the Medium unit. For the Large machine, the patient would need to be undressed but covering the eyes with UV protective goggles, necessary as well for the two smaller machines.

While treatment time would be determined experimentally, the beginning goal would be 20 minutes per day three times a week. For the Large, full body version of the device, this would mean 10 minutes on the front and 10 minutes on the back. The time of a session would be worked up to this maximum gradually to prevent skin burning.

The first distinctive feature of these machines and this method is the purpose. This is probably the first ultraviolet-based device and method designed specifically for treating cancer, including malignant tumors of the brain and central nervous system.

Second is its exclusive focus on only one of the three portions of sunlight. While the energy from sunlight is typically divided (from highest energy) into ultraviolet, visible light and infrared (heat), this device and method is careful to employ only one of the three portions—ultraviolet. Great care will be exercised to ensure that no energy from the infrared portion is included. As for visible light, this too would be excluded with the exception of a small portion to make sure that the human eye can discern when the unit is on.

Third, it is the only device specifically designed to mimic the intensity of ultraviolet in the major American city with the highest yearly value. Of course, the purpose here is to imitate conditions based on the statistical connection between the intensity of ultraviolet reaching the ground in that place over the year and the lowest incidence of malignant tumors of the brain and central nervous system.

1-34. (canceled)

35. The invention is a device for treating glioblastoma and other primary malignant tumors of the brain and central nervous system with ultraviolet radiation.

36. The said device in claim 35 is importantly defined by its two intensity values, its total intensity of between 6.6 to 9.6 as a UV Index score, and the intensity value in the UVB range of 3 watts per square meter.

37. The origin of the intensity values in claim 36 are as follows: 9.6 from the statistical research, the yearly UV Index value for Hawaii, 6.6 an average measured value in Honolulu between summer and winter, to simulate the value over the year, 3 watts per square meter UVB from several summer and winter values in Honolulu, 2.4 and 3.4 to average 2.9, rounded to 3.

38. The said device in claim 35 includes ultraviolet in both ranges which naturally reach the ground: UVB and UVA.

39. The said device in claim 35 includes wavelengths from 280 to 400 nanometers.

40. The said device in claim 35 consists of rows of florescent tubes emitting ultraviolet radiation, four in the small and medium versions and 6 in the large version.

41. The dimensions for the versions are as follows: small—22 inches wide, 9 inches deep and 14 inches high; medium—2 feet tall, one foot wide; large—to hold one row of five foot bulbs in a frame that can be tilted for use standing or lying.

42. The intensity values in claim 36 are achieved by positioning the said device in claim 35 the appropriate distance from the face or the area of the cancer in the head using the two appropriate solarmeter.com meters, the first a UV Index meter Model 6.5, and the second a UVB meter Model 6.0 (after converting milliwatts per centimeter square to watts per meter square).

43. The foundation for the device in claim 35 is a statistical correlation in a “big data” project linking the incidence of such malignant tumors by state in the United States to the insufficiently strong intensity of ultraviolet reaching the ground in sunlight in that state over the course of the year.

44. The said correlation in claim 42 is between two sets of numbers never before used in a patent application: “brain” cancer incidence numbers by state (2009-2013) from the Central Brain Tumor Registry of the United States and a special yearly version of the UV Index by state from the year 1996.

45. The size of the connection between these two things in said claim 43 is quite large—higher than 0.7, accounting for over half the pattern between states by itself, with ultraviolet accounting for more than a third of the total explanation.

46. The said invention in claim 35 is designed for exclusively treating cancer that has already taken hold and is not intended to be used for cancer prevention.

47. The said invention in claim 35 is unconnected with a cancer prevention strategy of vitamin D (and calcium) supplements which have shown no overall success in preventing cancer as of 2019 in a prospective study.

48. Treatment with ultraviolet as proposed here in claim 35 is largely separate from research on vitamin D in the blood 25(OH)D which includes the impact of vitamin D supplements which might not have the same effect as ultraviolet itself.

49. While the application here does not look at “inside the body” mechanisms, the chance exists that ultraviolet as proposed here in claim 35 might work entirely independently from vitamin D in an, as yet, unspecified way to fight cancer once it has taken hold.
